(19) United States
(12) Patent Application Publication
(10) Pub. No.: US 2021/0275374 A1
(43) Pub. Date: Sep. 9, 2021

(54) BED APPARATUS

(71) Applicant: PARAMOUNT BED CO., LTD., Tokyo (JP)

(72) Inventors: Yusuke SASAKI, Koto-ku Tokyo (JP); Kazuyuki OHHIRA, Koto-ku Tokyo (JP); Hiroki KIKUCHI, Koto-ku Tokyo (JP)

(21) Appl. No.: 17/259,435

(22) PCT Filed: Jul. 30, 2019
(86) PCT No.: PCT/JP2019/029760

§ 371 Date: Jan. 11, 2021

(30) Foreign Application Priority Data
Feb. 27, 2019 (JP) 2019-035002

(51) Int. Cl. A61G 7/05

(52) U.S. Cl. CPC A61G 7/0508; A61G 7/0524

(57) ABSTRACT

According to an embodiment, a bed apparatus includes a frame, a side rail, and a holder. The holder is secured to the frame. The holder holds the side rail to make the side rail switchable between a first position state where the side rail is in a first position and a second position state where the side rail is in a second position lower than the first position. The holder includes: a lock portion to lock the side rail in the first position state; a cover portion to cover at least part of the lock portion; a lever portion to be operated to release lock of the lock portion; and a stopper portion to make it difficult for the lever portion to operate to release the lock. The bed apparatus that can prevent erroneous release of the lock can be provided.

### TECHNICAL FIELD

An embodiment of the present invention relates to a bed apparatus.

### BACKGROUND ART

For example, a bed is provided with a side rail movable in a vertical direction. When the side rail is at an upper position, it is possible to prevent a user from falling from the bed. Meanwhile, when the side rail is at a lower position, it is possible to encourage the user to depart from the bed. A vertical movement of the side rail is locked to provide higher safety. It is desired to prevent erroneous release of the lock.

### RELATED ART DOCUMENTS

### Patent Documents

Patent Document 1: Japanese Unexamined Patent Application Publication No. 2008-29799.

### DISCLOSURE OF THE INVENTION

### Problems to be Solved by the Invention

The embodiment of the invention provides a bed apparatus that can prevent erroneous release of a lock.

### Means for Solving the Problems

According to the embodiment of the invention, a bed apparatus includes a frame, a side rail, and a holder. The holder is secured to the frame. The holder is configured to hold the side rail to make the side rail switchable between a first position state where the side rail is in a first position and a second position state where the side rail is in a second position lower than the first position. The holder includes: a lock portion configured to lock the side rail in the first position state; a cover portion configured to cover at least part of the lock portion; a lever portion configured to be operated to release lock of the lock portion; and a stopper portion configured to make it difficult for the lever portion to operate to release the lock.

### Effects of the Invention

According to the embodiment of the invention, it is possible to provide the bed apparatus that can prevent erroneous release of the lock.

### BRIEF DESCRIPTION OF THE DRAWINGS

FIG. 1(a) to FIG. 1(c) are schematic perspective views illustrating a bed apparatus according to a first embodiment.

FIG. 2(a) to FIG. 2(d) are schematic cross-sectional views illustrating the bed apparatus according to the first embodiment.

FIG. 3 is a schematic cross-sectional view illustrating the bed apparatus according to the first embodiment.

FIG. 4 (a) and FIG. 4 (b) are schematic perspective views illustrating a bed apparatus according to another embodiment.

FIG. 5(a) and FIG. 5(b) are schematic perspective views illustrating a bed apparatus according to another embodiment.

FIG. 6(a) and FIG. 6(b) are schematic perspective views illustrating a bed apparatus according to another embodiment.

FIG. 7 (a) and FIG. 7 (b) are schematic perspective views illustrating a bed apparatus according to another embodiment.

FIG. 8 (a) and FIG. 8 (b) are schematic perspective views illustrating a bed apparatus according to another embodiment.

FIG. 9 is a schematic perspective view illustrating a bed apparatus according to the embodiment.

FIG. 10(a) to FIG. 10(c) are schematic diagrams illustrating part of the bed apparatus according to the embodiment.

FIG. 11(a) and FIG. 11(b) are schematic diagrams illustrating part of the bed apparatus according to the embodiment.

FIG. 12(a) to FIG. 12(c) are schematic diagrams illustrating part of the bed apparatus according to the embodiment.

FIG. 13(a) and FIG. 13(b) are schematic diagrams illustrating part of the bed apparatus according to the embodiment.

FIG. 14 is a schematic diagram illustrating part of the bed apparatus according to the embodiment.

FIG. 15 is a schematic diagram illustrating part of the bed apparatus according to the embodiment.

FIG. 16(a) and FIG. 16(b) are schematic diagrams illustrating part of the bed apparatus according to the embodiment.

FIG. 17 (a) to FIG. 17 (c) are schematic perspective views illustrating operation of the bed apparatus according to the embodiment.

FIG. 18(a) and FIG. 18 (b) are schematic perspective views illustrating use states of the bed apparatus according to the embodiment.

FIG. 19 is a schematic diagram illustrating the bed apparatus according to the embodiment.

FIG. 20(a) and FIG. 20(b) are schematic diagrams illustrating part of the bed apparatus according to the embodiment.

FIG. 21(a) and FIG. 21(b) are schematic diagrams illustrating part of the bed apparatus according to the embodiment.

FIG. 22 is a schematic diagram illustrating part of the bed apparatus according to the embodiment.

FIG. 23 is a schematic perspective view illustrating part of the bed apparatus according to the embodiment.

FIG. 24 is a schematic diagram illustrating part of the bed apparatus according to the embodiment.

FIG. 25(a) and FIG. 25(b) are schematic diagrams illustrating part of the bed apparatus according to the embodiment.

### MODE FOR CARRYING OUT THE INVENTION

Hereinafter, each embodiment of the invention will be described with reference to the drawings.

In the description of this application and each of the drawings, substantially the same elements as those already described with reference to a preceding drawing are denoted by identical reference numerals and signs and will not be elaborated as appropriate.

### First Embodiment

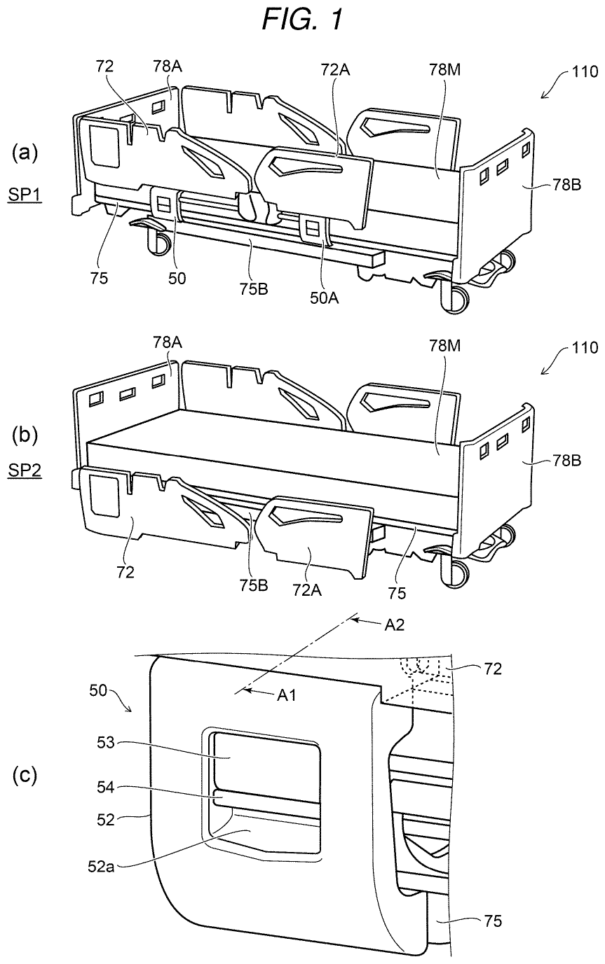

FIG. 1(a) to FIG. 1(c) are schematic perspective views illustrating a bed apparatus according to a first embodiment.

FIG. 1(a) and FIG. 1(b) illustrate different states of a bed apparatus 110. FIG. 1(c) illustrates part of the bed apparatus 110 in an enlarged view.

As illustrated in FIG. 1(a), the bed apparatus 110 according to the embodiment includes a frame 75, a side rail 72, and a holder 50. The holder 50 holds the side rail 72.

In an example of FIG. 1(a), a plurality of side rails (the side rail 72 and a different side rail 72A) and a plurality of holders (the holder 50 and a different holder 50A) are provided. The different side rail 72A may be substantially the same as the side rail 72 except for a size and a disposed position. The different holder 50A holds the different side rail 72A. As illustrated in FIG. 1(a), the side rail 72 and the holder 50, for example, may be provided on either side of the left and the right of the bed. In the following, the single side rail 72 and the single holder 50 will be described.

The frame 75 is secured to a base frame 75B, for example. Sections (not illustrated) are disposed on the frame 75, and a mattress 78M is disposed on the sections. A user of the bed apparatus 110 can lie on the mattress 78M.

For example, the bed apparatus 110 includes a headboard 78A and a footboard 78B. A direction from the headboard 78A to the footboard 78B corresponds to a “longitudinal direction”. A direction that intersects the “longitudinal direction” corresponds to a “width direction”. The “width direction” corresponds to, for example, a “lateral direction” when the user is asleep on the mattress 78M.

The frame 75 extends in the “longitudinal direction”, for example.

For example, the holder 50 is secured to the frame 75. One portion of the holder 50 is secured to the frame 75. A different portion of the holder 50 is coupled to the side rail 72. Thus, the holder 50 holds the side rail 72.

In the embodiment, the holder 50 holds the side rail 72 in a plurality of states. FIG. 1(a) illustrates a first position state SP1. FIG. 1(b) illustrates a second position state SP2. In the first position state SP1, the side rail 72 is in a first position. In the second position state SP2, the side rail 72 is in a second position. The second position is lower than the first position. In this manner, the holder 50 holds the side rail 72 in such a manner that the side rail 72 can be switched between the first position state SP1 and the second position state SP2. A position of an upper end of the side rail 72 in the first position state SP1 (a raised state) is higher than a position of an upper surface of the mattress 78M. A position of the upper end of the side rail 72 in the second position state SP2 (a lowered state) is lower than the position of the upper surface of the mattress 78M.

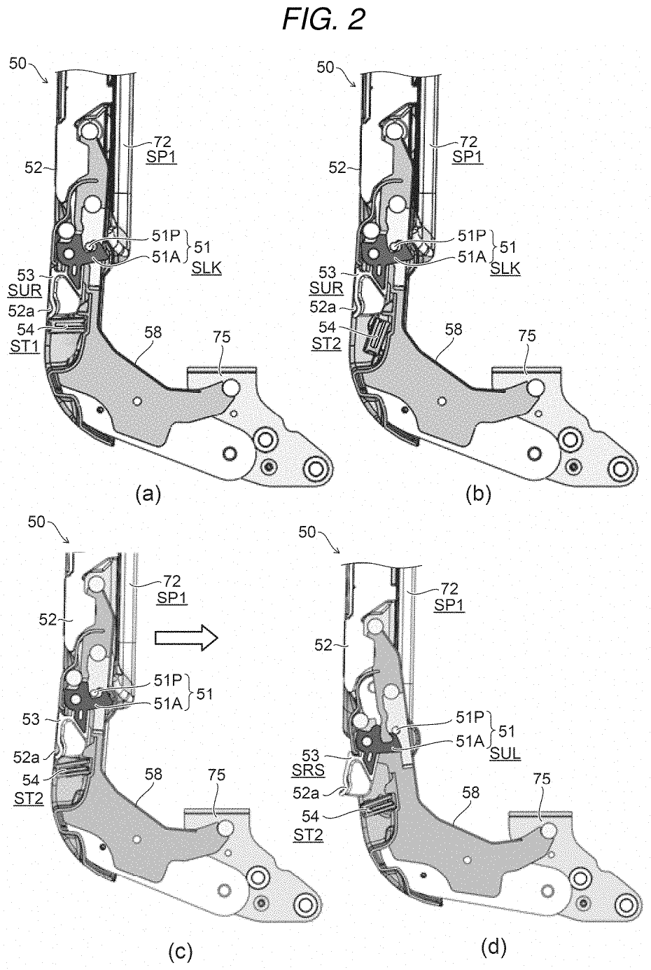

As illustrated in FIG. 1(c), the holder 50 includes a cover portion 52, a lever portion 53, and a stopper portion 54. The holder 50 further includes a lock portion 51 (see, for example, FIG. 2(a)), described later. In this example, the cover portion 52 includes a recess 52a. At least part of the lever portion 53 is disposed in the recess 52a of the cover portion 52.

In this example, at least part of the stopper portion 54 is disposed in the recess 52a. The at least part of the stopper portion 54 is disposed in the recess 52a and below the lever portion 53.

The lock portion 51 locks the side rail 72 in the first position state SP1. The cover portion 52 covers at least part of the lock portion 51. The lever portion 53 is operated to release the lock of the lock portion 51. The stopper portion 54 makes it difficult for the lever portion 53 to operate to release the lock.

Hereinafter, examples of these portions of the holder 50 will be described.

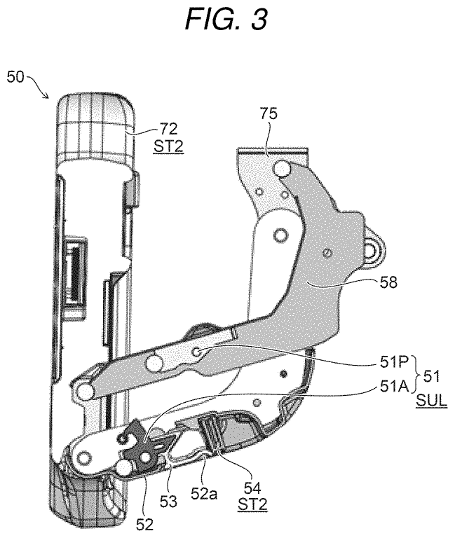

FIG. 2(a) to FIG. 2(d) and FIG. 3 are schematic cross-sectional views illustrating the bed apparatus according to the first embodiment.

These drawings are cross-sectional views corresponding to cross sections taken along line A1-A2 in FIG. 1(c).

As illustrated in FIG. 2 (a), the holder 50 includes the lock portion 51, the cover portion 52, the lever portion 53, and the stopper portion 54. In FIG. 2(a), the side rail 72 is in the first position state SP1 (a raised state).

The lock portion 51 has a locked state SLK (see FIG. 2 (a)) and an unlocked state SUL (see FIG. 2 (d)). When the lock portion 51 is in the locked state SLK, the lock portion 51 locks the side rail 72 in the first position state SP1. In this example, the lock portion 51 includes a hook 51A and a pin 51P. The pin 51P is secured to a lock component 58. In one state (the locked state SLk) of the hook 51A, the hook 51A is hooked by the pin 51P. Thus, the lock portion 51 locks the side rail 72 in the first position state SP1.

As illustrated in FIG. 2(a), the cover portion 52 covers at least part (front surface part) of the lock portion 51. Since the cover portion 52 covers a front surface of the lock portion 51, cleanability can be increased, and an internal mechanism can be concealed.

As already described, at least part of the lever portion 53 is disposed in the recess 52a of the cover portion 52. A user of the bed apparatus 110 or a care worker, for example, can touch the lever portion 53.

The lever portion 53 can have a plurality of states. The plurality of states include, for example, a non-released state SUR (see FIG. 2 (a)) and a released state SRS (see FIG. 2(d)). For example, when the lever portion 53 is in the non-released state SUR (see FIG. 2(a)), the lock portion 51 is in the locked state SLK. When the lever portion 53 is in the non-released state SUR, the lock portion 51 does not shift to the unlocked state SUL. As described later, when the lever portion 53 is in the released state SRS (see FIG. 2(d)), the side rail 72 can shift to the unlocked state SUL.

For example, the stopper portion 54 can also have a plurality of states. The plurality of states include a first state ST1 (see FIG. 2(a)) and a second state ST2 (see FIG. 2(b)). When the stopper portion 54 is in the first state ST1 (see FIG. 2(a)), the stopper portion 54 makes it difficult for the lever portion 53 to shift to the released state SRS. When the stopper portion 54 is in the second state ST2 (see FIG. 2(b)), the stopper portion 54 makes it possible for the lever portion 53 to shift to the released state SRS (see FIG. 2(d)). The first state ST1 is a lock release difficult state (or a lock release inhibition state). The second state ST2 is a lock release shiftable state.

It is more difficult for the lever portion 53 to shift to the released state SRS when the stopper portion 54 is in the first state ST1 than for the lever portion 53 to shift to the released state SRS when the stopper portion 54 is in the second state ST2. The lever portion 53 shifts to the released state SRS more easily when the stopper portion 54 is in the second state ST2 than when the stopper portion 54 is in the first state ST1.

For example, in a state illustrated in FIG. 2(a), the lever portion 53 is in the non-released state SUR. Then, the stopper portion 54 is in the first state ST1. In this example, the stopper portion 54 is in a raised state so that the stopper portion 54 is substantially in contact with a lower portion of the lever portion 53. Since the stopper portion 54 is substantially in contact with the lower portion of the lever portion 53, fingers of the user, for example, can be prevented from erroneously entering space between the stopper portion 54 and the lever portion 53. When the stopper portion 54 is in the first state ST1, the user can be consequently hindered from operating the lever portion 53.

As illustrated in FIG. 2(b), for example, the stopper portion 54 can be moved in a downward direction by, for example, a care worker's operation. For example, an end of the stopper portion 54 is turned about one shaft serving as a center so that the stopper portion 54 shifts to a lowered state. This state corresponds to the second state ST2 of the stopper portion 54.

In the second state ST2, a gap is formed between the lever portion 53 and the stopper portion 54. This gap has such a width (a distance in the vertical direction) that fingers of the care worker may enter the gap. The care worker, for example, inserts the care worker's hand into this gap so as to touch the lower portion of the lever portion 53. In the second state ST2 of the stopper portion 54 (see FIG. 2(b)), the lever portion 53 becomes capable of shifting to the released state SRS (see FIG. 2 (d)) to release the locked state SLK of the lock portion 51.

In this example, a different state (a state of FIG. 2 (c)) is provided between the state of FIG. 2(b) and the state of FIG. 2(d). As illustrated in FIG. 2(c), after the stopper portion 54 shifts to the second state ST2, the holder 50 (or the side rail 72) is pressed toward the mattress 78M so that the hook 51A of the lock portion 51 is easily detached from the pin 51P.

After this, as illustrated in FIG. 2(d), an end of the lever portion 53 is raised in an upward direction by, for example, the care worker's operation. For example, a front end of the lever portion 53 is turned about a rear shaft of the lever portion 53. Thus, the hook 51A of the lock portion 51 is detached from the pin 51P. The lock portion 51 shifts to the unlocked state SUL. In this manner, by operating the lever portion 53, the lever portion 53 shifts to the released state SRS, and the lock portion 51 is released from the locked state SLK.

As illustrated in FIG. 3, when the locked state SLK of the lock portion 51 is released, the side rail 72 can be consequently moved in the downward direction. This state corresponds to the second position state SP2.

In this manner, the embodiment has the first state ST1, which is the lock release difficult (or inhibited) state of the stopper portion 54, and the second state ST2, which is the lock release shiftable state of the stopper portion 54. Thus, for example, a person such as the user of the bed apparatus 110 can be prevented from erroneously releasing the lock. According to the embodiment, the bed apparatus that can prevent erroneous release of the lock can be provided.

In the above-described example, the state where the stopper portion 54 is moved in the downward direction (see FIG. 2 (b)) is followed by the state where the holder 50 (or the side rail 72) is pressed toward the mattress 78M (see FIG. 2(c)). In the embodiment, after the holder 50 (or the side rail 72) is pressed toward the mattress 78M, the stopper portion 54 may be moved in the downward direction.

In the embodiment, a distance between at least part of the stopper portion 54 when the stopper portion 54 is in the first state ST1 and at least part of the lever portion 53 is shorter than a distance between the at least part of the stopper portion 54 when the stopper portion 54 is in the second state ST2 and the at least part of the lever portion 53.

For example, when the stopper portion 54 is in the first state ST1, a distance between the stopper portion 54 and the lever portion 53 is, for example, 8 mm or less. Thus, in the first state ST1, fingers, for example, can be prevented from erroneously entering the space between the stopper portion 54 and the lever portion 53. As a result, the first state ST1 to maintain the locked state is stabilized.

When the stopper portion 54 is in the second state ST2, a distance between the stopper portion 54 and the lever portion 53 exceeds, for example, 8 mm. Thus, in the second state ST2, fingers, for example, can more easily enter the space between the stopper portion 54 and the lever portion 53 so as to facilitate operation of the lever portion 53.

### Other Embodiments

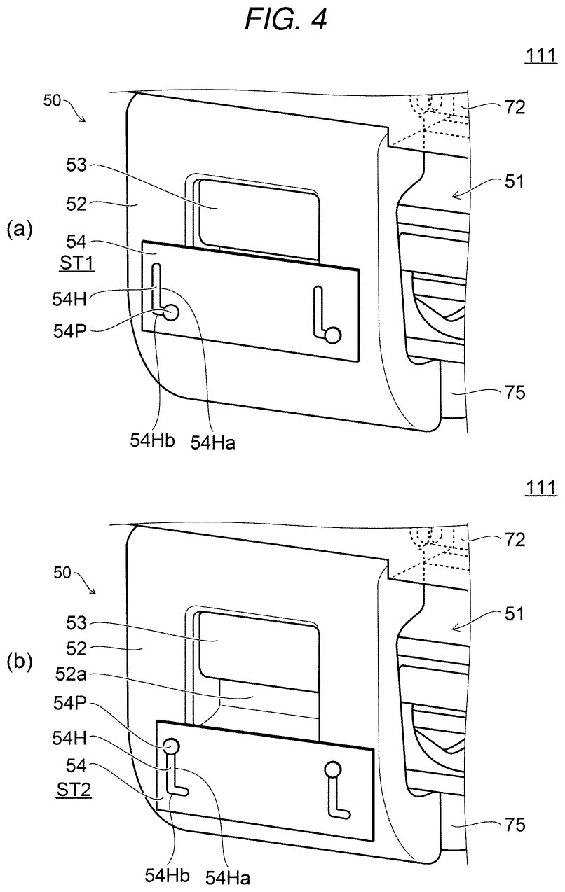

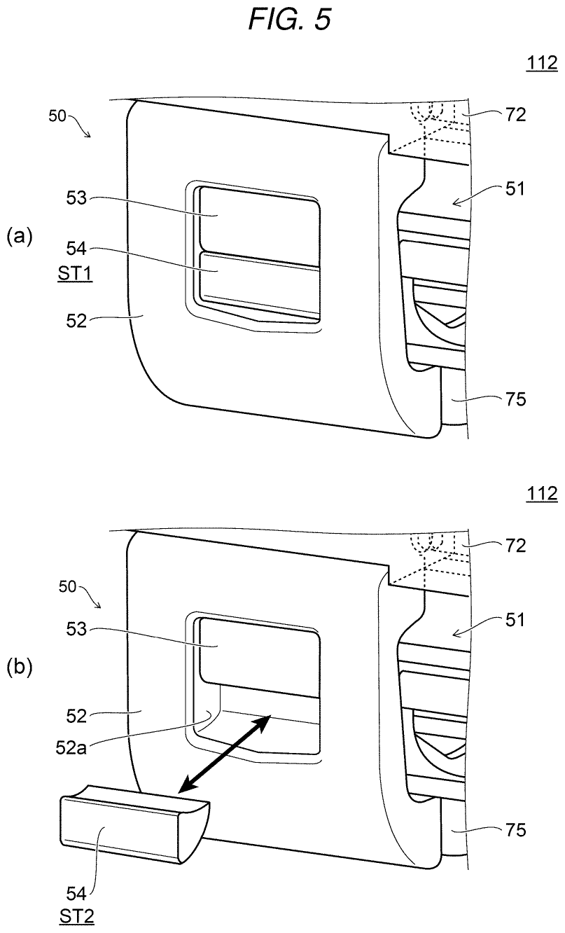

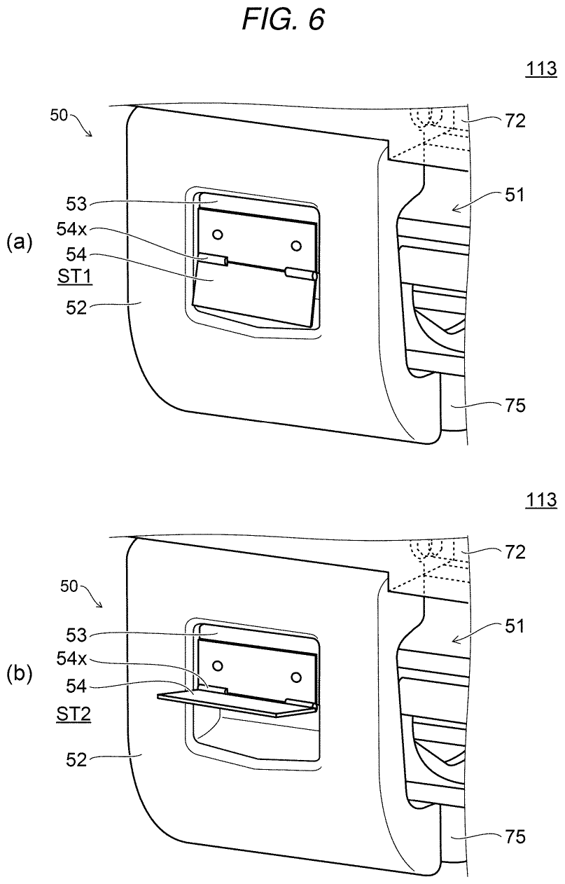

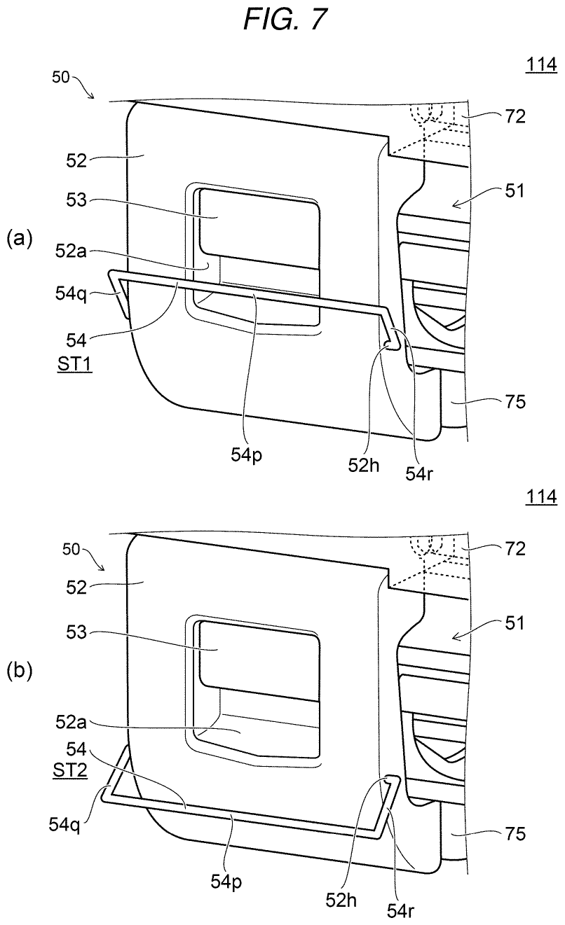

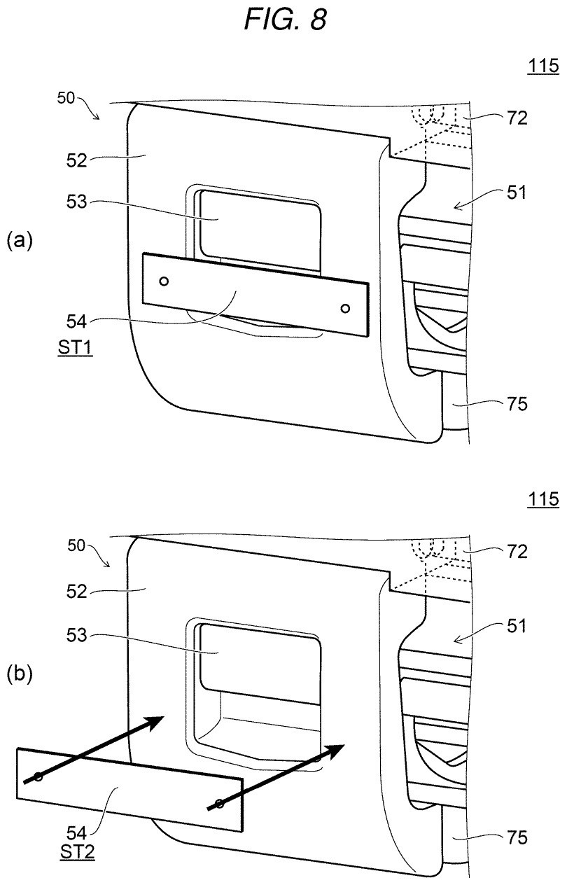

FIG. 4(a), FIG. 4(b), FIG. 5(a), FIG. 5(b), FIG. 6(a), FIG. 6(b), FIG. 7(a), FIG. 7(b), FIG. 8(a), and FIG. 8(b) are schematic perspective views illustrating bed apparatuses according to other embodiments.

These drawings illustrate the holder 50. Those portions except the holder 50 are as already described with reference to FIG. 1 (a) and FIG. 1 (b). In these drawings, the lock portion 51, which is covered with the cover portion 52, cannot be seen.

In a bed apparatus 111 illustrated in FIG. 4 (a) and FIG. 4(b), the stopper portion 54 includes a plate-shaped portion. The plate-shaped portion (the stopper portion 54) has a hole 54H. A first portion 54Ha of the hole 54H extends in a first direction (e.g., a gravity direction). A second portion 54Hb of the hole 54H extends in a direction intersecting the first direction (e.g., a direction along the frame 75). A position of the second portion 54Hb is below a position of an upper end of the first portion 54Ha. A pin 54P is inserted in the hole 54H.

In FIG. 4 (a), the stopper portion 54 is in the first state ST1 (the lock release difficult (inhibited) state). At this time, the pin 54P is in the second portion 54Hb of the hole 54H. Thus, an upper end of the stopper portion 54 is fixed in a state close to a lower end of the lever portion 53. The stopper portion 54 hinders the lever portion 53 from being operated.

In FIG. 4(b), the stopper portion 54 is in the second state ST2 (the lock release shiftable state). At this time, the pin 54P is in an upper end of the first portion 54Ha of the hole 54H. Thus, the upper end of the stopper portion 54 is separated from the lower end of the lever portion 53. As a result, the lever portion 53 can be operated.

In a bed apparatus 112 illustrated in FIG. 5(a) and FIG. 5(b), the stopper portion 54 includes a filling member. The filling member (the stopper portion 54) can fill a space in the recess 52a of the cover portion 52 and below the lever portion 53.

In FIG. 5 (a), the stopper portion 54 is in the first state ST1 (the lock release difficult (inhibited) state). At this time, the filling member (the stopper portion 54) is inserted in the space in the recess 52a of the cover portion 52 and below the lever portion 53. The filling member (the stopper portion 54) hinders the lever portion 53 from being operated.

In FIG. 5(b), the stopper portion 54 is in the second state ST2 (the lock release shiftable state). At this time, the filling member (the stopper portion 54) is removed from the recess 52a of the cover portion 52. Thus, the lever portion 53 can be operated.

In a bed apparatus 113 illustrated in FIG. 6(a) and FIG. 6(b), the stopper portion 54 is in the form of a hinge. One side of the hinge is attached to the lever portion 53. The other side of the hinge corresponds to the stopper portion 54. The one side of the hinge and the other side of the hinge are coupled in a rotatable state about shafts 54x.

In FIG. 6(a), the stopper portion 54 is in the first state ST1 (the lock release difficult (inhibited) state). At this time, the stopper portion 54 blocks up the space in the recess 52a of the cover portion 52 and below the lever portion 53. The stopper portion 54 hinders the lever portion 53 from being operated.

In FIG. 6(b), the stopper portion 54 is in the second state ST2 (the lock release shiftable state). At this time, the stopper portion 54 is rotated in the upward direction so that the lower portion of the lever portion 53 can be touched. Thus, the lever portion 53 can be operated.

In a bed apparatus 114 illustrated in FIG. 7 (a) and FIG. 7(b), the stopper portion 54 includes a bar-shaped member 54p. The bar-shaped member 54p extends, for example, in the direction along the frame. One end of the bar-shaped member 54p is coupled to a first member 54q. The other end of the bar-shaped member 54p is coupled to a second member 54r. 8 of each of the first member 54q and the second member 54r is inserted in a hole 52h formed in the cover portion 52, and is rotatably supported.

In FIG. 7 (a), the stopper portion 54 is in the first state ST1 (the lock release difficult (inhibited) state). At this time, the bar-shaped member 54p is in front of the space below the lever portion 53. The bar-shaped member 54p hinders the lever portion 53 from being operated.

In FIG. 7 (b), the stopper portion 54 is in the second state ST2 (the lock release shiftable state). At this time, the bar-shaped member 54p is rotated in the downward direction so that the lower portion of the lever portion 53 can be touched. Thus, the lever portion 53 can be operated.

In a bed apparatus 115 illustrated in FIG. 8 (a) and FIG. 8(b), the stopper portion 54 is detachable from the cover portion 52. For example, the stopper portion 54 can be made detachable from the cover portion 52 by any of mechanical, electrical, and magnetic methods. For example, the stopper portion 54 is attached to the cover portion 52 with a component such as pins. The attachment may be performed with a planar fastener, for example. The attachment may be performed with static electricity, for example. The attachment may be performed with a magnet, for example.

In FIG. 8 (a), the stopper portion 54 is in the first state ST1 (the lock release difficult (inhibited) state). At this time, the stopper portion 54 is in front of the space below the lever portion 53. The stopper portion 54 hinders the lever portion 53 from being operated.

In FIG. 8(b), the stopper portion 54 is in the second state ST2 (the lock release shiftable state). At this time, the stopper portion 54 is detached. Thus, the lever portion 53 can be operated.

In the embodiments, configurations of the holder 50 (configurations of the lock portion 51, the cover portion 52, the lever portion 53, and the stopper portion 54) can be modified in various manners.

In the above-described bed apparatuses 110 to 115, part of the lever portion 53 may be outside of the recess 52a in at least one of the non-released state SUR, the released state SRS, and a state between the non-released state SUR and the released state SRS (see, for example, FIG. 2 (d)). In this case, as in the bed apparatus 110 or 112, at least part of the stopper portion 54 is in the recess 52a so as to further prevent restriction of an operation state of the lever portion 53.

Hereinafter, an exemplary bed apparatus according to the embodiment will be described.

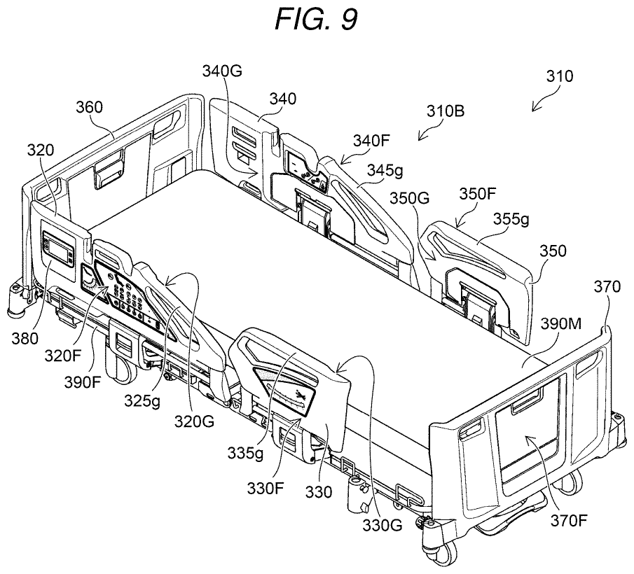

FIG. 9 is a schematic perspective view illustrating the bed apparatus according to the embodiment.

As illustrated in FIG. 9, a bed apparatus 310 includes a head right side rail 320, a foot right side rail 330, a head left side rail 340, a foot left side rail 350, a headboard 360, and a footboard 370.

These side rails are disposed on, for example, a frame 390F of a bed 310B. The sections (not illustrated in FIG. 9) are disposed on the frame 390F of the bed 310B, and a mattress 390M is disposed on the sections. A user of the bed 310B can lie on the mattress 390M. The bed apparatus 310 is used in, for example, a hospital, a care facility, or a household.

The bed apparatus 310 is, for example, a motorized bed. The bed apparatus 310 can be operated by the user of the bed apparatus 310, a care worker or the like.

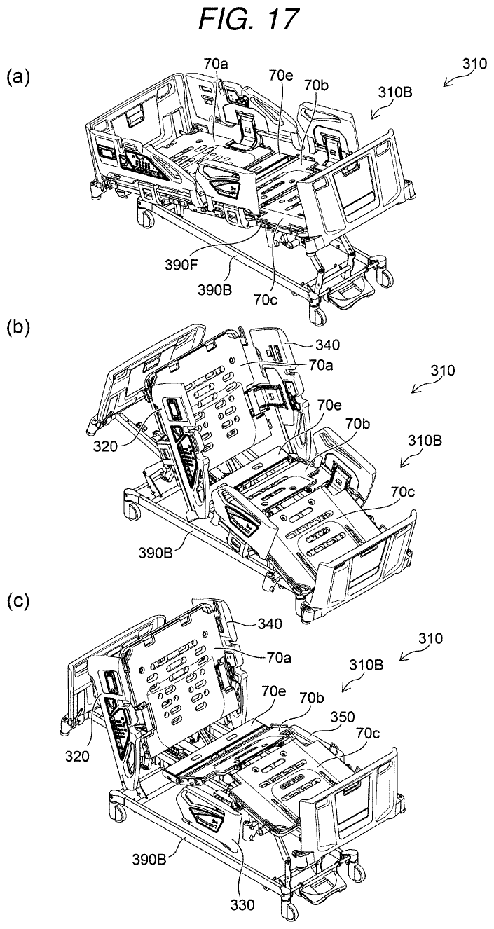

Angles of the head right side rail 320 and the head left side rail 340 (e.g., angles with respect to a horizontal plane as a reference) are variable. For example, back raising or back lowering can be performed. For example, when an angle of the back section 70a (see FIG. 17(b)) is changed, the angles of the head right side rail 320 and the head left side rail 340 attached to the back section 70a are changed to follow the back section 70a.

Heights of the foot right side rail 330 and the foot left side rail 350 are variable. When the foot right side rail 330 and the foot left side rail 350 are at high positions, for example, the user can be prevented from falling from the bed 310B. When the foot right side rail 330 and the foot left side rail 350 are at low positions, for example, the user easily departs from the bed 310B through an upper side of the foot right side rail 330 and the foot left side rail 350.

The head right side rail 320 has an outer surface 320F and an inner surface 320G. The foot right side rail 330 has an outer surface 330F and an inner surface 330G. The head left side rail 340 has an outer surface 340F and an inner surface 340G. The head left side rail 340 has an outer surface 350F and an inner surface 350G.

Various switches and the like are disposed on the outer surface 320F and the inner surface 320G of the head right side rail 320 and the outer surface 340F and the inner surface 340G of the head left side rail 340. The switches on the outer surfaces are designed to be convenient, for example, for a care worker or a medical worker (such as a medical doctor, a nurse, or a physical therapist) to operate. Various switches and the like are also disposed on these inner surfaces. The switches on the inner surfaces are designed to be convenient, for example, for the user of the bed 310B. Examples of these switches will be described later.

Handrails 325g, 335g, 345g, and 355g are disposed on respective upper portions of the head right side rail 320, the foot right side rail 330, the head left side rail 340, and the foot left side rail 350. Widths of these handrails in the vertical direction are smaller on an inner side than on an outer side. This, for example, makes it easier for the user to grasp these handrails.

In the foot right side rail 330 and the foot left side rail 350, upper surfaces of the respective handrails 335g and 355g are designed to have large widths. The user can sit on these upper surfaces (edge sitting position). Thus, when the user is in an edge sitting position, back sides of the thighs are prevented from aching.

Recesses are formed in the outer surface 320F of the head right side rail 320 and the outer surface 340F of the head left side rail 340. Abed user interface device 380 can be attached to these recesses. Moreover, the bed user interface device 380 can be attached to an outer surface 370F of the footboard 370. The bed user interface device 380 will be described later.

Hereinafter, examples of the side rails will be described.

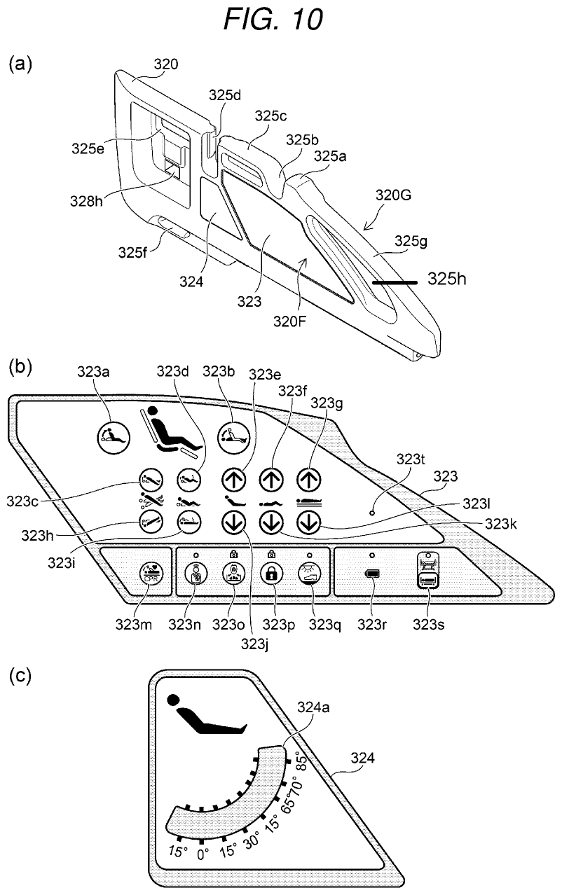

FIG. 10(a) to FIG. 10(c) are schematic diagrams illustrating part of the bed apparatus according to the embodiment.

As illustrated in FIG. 10(a), a through hole 325h is formed in an upper portion of the head right side rail 320. The through hole 325h forms the handrail 325g.

The upper portion of the head right side rail 320 further includes a protrusion 325a, a recess 325b, a head-side protrusion 325c, and a head-side recess 325d. The protrusion 325a can be used as, for example, a support portion to support the body of the user. When the head right side rail 320 is raised (a back raised state), the recess 325b can be used as, for example, a support portion to support the body of the user.

The user easily grasps the head-side protrusion 325c. For example, in a back raised state or a back lowered state, the body of the user is easily supported by the head-side protrusion 325c. The head-side protrusion 325c has a though hole. Thus, the head-side protrusion 325c can be used as a handrail.

The head-side recess 325d can contain various lines for medical care. The various lines are easily made stable. The various lines for medical care include, for example, cables or tubes of a respirator and various kinds of ME equipment. The various lines are inserted in the head-side recess 325d so that these lines can be prevented from being tangled.

The outer surface 320F of the head right side rail 320 is provided with a switch portion 323, an angle meter 324, a through hole 325e (e.g., a hook portion), and a lower through hole 325f (e.g., a Harn bag hook). The bed user interface device 380 can be hung from the through hole 325e. A Harn bag can be hung from the lower through hole 325f. A trash box, for example, may be hung from the lower through hole 325f. For example, a hole 328h is formed below the through hole 325e. A cable of the bed user interface device 380 may be passed through the hole 328h. The cable is electrically connected to a connector disposed on the bed apparatus 310 via the hole 328h. The cable can be prevented from coming into contact with a floor. The cable can be shortened.

FIG. 10(b) illustrates the switch portion 323.

The switch portion 323 is, for example, a membrane switch (such as a membrane switch for medical workers). The switch portion 323 includes switches 323a to 323q.

When the switch 323a is depressed, “cardiac raising” is performed. When the switch 323b is depressed, “cardiac lowering” is performed. The “cardiac raising” causes the bed 310B to take a cardiac position (see FIG. 17 (b)). By the “cardiac raising”, the bed 310B is operated toward the cardiac position without decreasing an angle between the back section 70a and the upper leg section 70b to an extreme degree while keeping the upper leg section 70b raised from a horizontal angle. In the state where the angle between the back section 70a and the upper leg section 70b is not extremely decreased, the angle is less than 90 degrees. In the state where the angle between the back section 70a and the upper leg section 70b is not extremely decreased, the user can be prevented from receiving excessive abdominal pressure and can keep a comfortable posture. For example, when the user is inclined without raising the knees, the user may slip down in some cases. With the upper leg section 70b being raised from the horizontal angle, the user can be prevented from slipping down. By the “cardiac lowering”, the bed 310B is operated into a state with aback angle of 0 degree, a knee angle of 0 degree, and an inclination angle of 0 degree without decreasing an angle between the back section 70a and the upper leg section 70b to an extreme degree while keeping the upper leg section 70b raised from the horizontal angle.

When the switch 323c is depressed, “reverse expansion” is performed. When the switch 323h is depressed, “expansion” is performed. In the “expansion”, the head is lowered. In the “reverse expansion”, the feet are lowered.

When the switch 323d is depressed, “kind raising” is performed. In the “kind raising”, angles of the back section 70a and the upper leg section 70b vary in conjunction with each other. When the switch 323i is depressed, “kind lowering” is performed. In the “kind lowering”, the angles of these sections vary in conjunction with each other. For example, when the angles of the back section 70a and the upper leg section 70b increase in conjunction with each other, the angle of the upper leg section 70b increases first, and thereafter, the angle of the other section increases. This minimizes displacement of a patient (user). In the “kind raising”, the angle of the back section 70a being 70 degrees corresponds to an operation completion angle. The angle of the upper leg section 70b is an angle to follow the angle of the back section 70a.

In the “kind raising”, at first, the back angle is 0 degree, and the knee angle is 0 degree. Next, the back angle becomes 5 degrees, and the knee angle is 0 degree. Next, the back angle becomes 15 degrees, and the knee angle becomes 10 degrees. Further, the back angle becomes 30 degrees, the knee angle becomes 25 degrees, the back angle becomes 50 degrees, and the knee angle becomes 25 degrees. Thereafter, the back angle becomes 70 degrees, and the knee angle becomes 0 degree. In the “kind raising”, the back angle and the knee angle increase in conjunction with each other part way. When the back angle becomes a certain angle or larger, the knee angle decreases to 0 degree.

In the “kind lowering”, at first, the back angle is 70 degrees, and the knee angle is 0 degree. Next, the back angle becomes 50 degrees, and the knee angle becomes 25 degrees. Further, the back angle becomes 30 degrees, and the knee angle is 25 degrees. Further, the back angle becomes 15 degrees, and the knee angle becomes 20 degrees. Further, the back angle becomes 0 degree, and the knee angle becomes 5 degrees. Thereafter, the back angle is 0 degree, and the knee angle becomes 0 degree.

When the switch 323e is depressed, “back raising” is performed. When the switch 323j is depressed, “back lowering” is performed.

When the switch 323f is depressed, “knee raising” is performed. When the switch 323k is depressed, “knee lowering” is performed.

When the switch 323g is depressed, “height raising” is performed. When the switch 323l is depressed, “height lowering” is performed. When a height of the bed 310B in an inclined state is raised, the height is raised while an inclination angle is maintained. The same applies to the “lowering”. Further, when the bed 310B reaches a maximum bed height while keeping the inclined state, the bed 310B stops once. After the bed 310B stops, and when a height raising button (the switch 323g) is depressed again, priority is given to raising the height of the bed 310B, and the height is raised while the inclination angle is made to be 0 degree. The same applies to the “lowering”.

The switches 323c to 323g for “raising” are above the switches 323h to 3231 for “lowering”. For example, there is a possibility that the user of the bed 310B unintentionally touches the switches of the switch portion 323. At this time, the user of the bed 310B is more likely to touch an upper portion than a lower portion. The switches 323c to 323g for “raising” are on an upper side so that even when the user erroneously touches the upper portion, a risk can be suppressed as compared with the case of touching the switches 323h to 323l.

When the switch 323m is depressed, “CPR lowering” is performed. In the “CPR lowering”, the bed is in a state suitable for cardio pulmonary resuscitation (CPR). In the “CPR lowering” state, the upper leg section 70b and the lower leg section 70c become flat. In the “CPR lowering”, the bed height of the bed 310B is lowered. In the case of an inclined state, an inclination angle also becomes 0 degree. For example, an operation sequence is as follows. The back angle is made to be 0 degree (while the back section 70a is being moved, the upper leg section is also made to be close to 0 degree). Next, the inclination angle is made to be 0 degree. Next, the height is lowered. Next, the knee angle is made to be 0 degree. For example, the bed 310B may be in a state where the bed height is lowest. For example, the bed 310B may be provided with a temporary stop position (a temporary stop height) by the operation of “height lowering”. In a state prior to the “CPR lowering”, when the height is larger than the temporary stop height, the “CPR lowering” is performed to cause the height of the bed 310B to be the temporary stop height. At the temporary stop height, a distance from the floor to upper surfaces of the sections is approximately 42 cm.

In one example, when the switch 323m is “depressed and held”, the “CPR lowering” is performed. Time of “depressing and holding” is 2 seconds or longer, for example. In another example, when the switch 323m is “depressed twice”, the “CPR lowering” is performed. When the switch 323m is “depressed twice”, time between “first depressing” and “second depressing” is 5 seconds or less.

Concerning operations other than CPR operations, an operation is started when a button is depressed. In an emergency, to quickly perform a CPR operation, an operator of the button for the CPR operation (switch 323m) may hesitate to depress the button for the CPR operation. As described above, for example, the CPR operation is performed by “depressing and holding” or “depressing twice” the switch 323m. Thus, the CPR operation can be performed with less hesitation.

The “CPR lowering” is an electric CPR operation. Other than this, a manual CPR operation may be performed.

The switch 323n is a “nurse call”. When the switch 323n is depressed, nurse call dispatch is performed. Information is transmitted to a nurse call system.

When the switch 323o is depressed, an operation of a switch (described later) disposed on the inner surface 320G of the head right side rail 320 shifts to an “inhibited state”. When the switch 323p is depressed, all operations concerning movements (actuators) of the bed 310B shift to “inhibited states”. In this case, the “nurse call” and a foot lamp, for example, can be operated.

When the switch 323q is depressed, the foot lamp provided for the bed 310B, for example, is lit.

In this example, the switch portion 323 includes indicators 323r to 323t. The indicator 323r indicates a remaining capacity of a battery. The indicator 323s is lit (e.g., orange colored) when the bed height is not the minimum. The indicator 323s is unlit when the bed height is the minimum.

The indicator 323t displays an error. Normally, the indicator 323t is unlit. At the time of “U-system abnormality”, the indicator 323t is lit for 1 second and unlit for 1 second repeatedly. At the time of “H-system abnormality”, the indicator 323t is repeatedly lit for 0.2 second and unlit for 0.2 second.

Operation by the switch portion 323 (e.g., membrane switches for medical workers) on the outer surface 320F has priority to operation by a switch portion (e.g., membrane switches for the user), described later, disposed on the inner surface 320G. For example, when the outer and inner switches are simultaneously depressed, neither operation is performed. For example, when back raising is performed by the outer switch, and when the inner knee raising button is depressed, the bed 310B is stopped. When both of the buttons are released and depressed again, the bed 310B is operated.

FIG. 10(c) illustrates the angle meter 324 disposed on the outer surface 320F of the head right side rail 320.

A recess is formed in the head right side rail 320, and a sphere (such as a metal sphere) is disposed in the recess so as to constitute the angle meter 324. When the back section is raised, the sphere rolls in the recess. In accordance with a position of the sphere, angle display in a display 324a of the angle meter 324 varies. An overview of the back angle can be known from the angle meter 324.

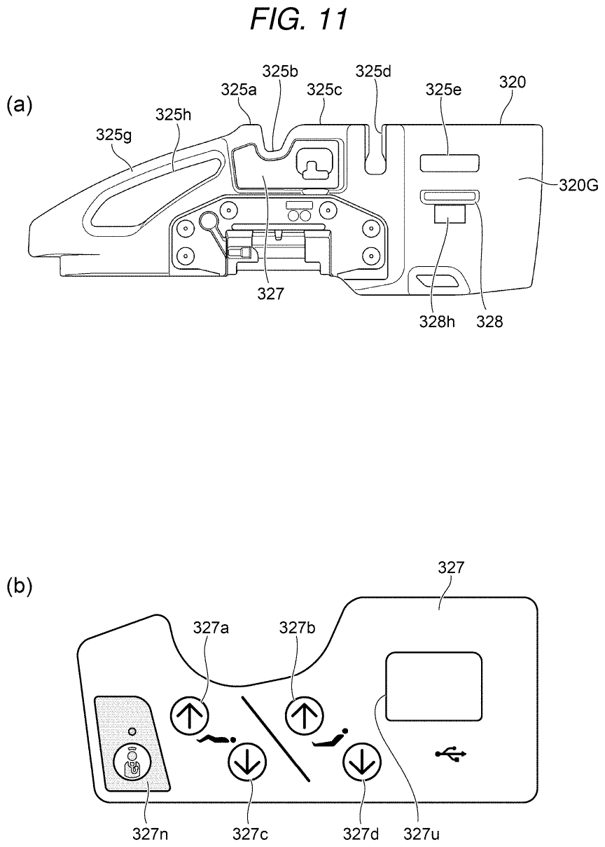

FIG. 11(a) and FIG. 11(b) are schematic diagrams illustrating part of the bed apparatus according to the embodiment.

As illustrated in FIG. 11 (a), a recess 328 is formed in the inner surface 320G of the head right side rail 320. The recess 328 can be used as a hook. The hole 328h is formed below the recess 328. Further, a switch portion 327 is disposed on the inner surface 320G. The switch portion 327 is, for example, a membrane switch (e.g., a membrane switch for the user or a membrane switch for the patient).

As illustrated in FIG. 11(b), the switch portion 327 includes switches 327a to 327d. The switch portion 327 may include a switch 327n.

When the switch 327a is depressed, “foot raising” is performed. When the switch 327c is depressed, “foot lowering” is performed.

When the switch 327b is depressed, “back raising” is performed. When the switch 327d is depressed, “back lowering” is performed.

The switch 327n is disposed on the inner surface 320G. The switch 327n is “nurse call”.

A USB terminal 327u is disposed on the inner surface 320G. A USB plug can be inserted into the USB terminal 327u to perform charging, for example.

The above-described configuration of the head right side rail 320 is also applied to the head left side rail 340.

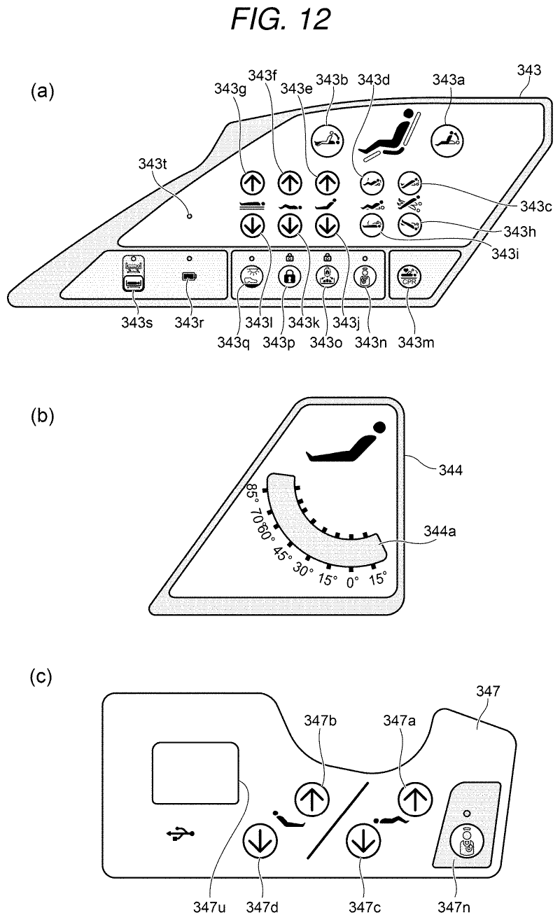

FIG. 12(a) to FIG. 12(c) are schematic diagrams illustrating part of the bed apparatus according to the embodiment.

FIG. 12(a) and FIG. 12(b) illustrate a switch portion 343 and an angle meter 344 that are disposed on an outer surface 340F of the head left side rail 340. As illustrated in FIG. 12 (a), the switch portion 343 includes switches 343a to 343q. The switches 343a to 343q have substantially the same functions as the switches 323a to 323q. In this example, the switch portion 343 includes indicators 343r to 343t. The indicators 343r to 343t have substantially the same functions as the indicators 323r to 323t.

FIG. 12(b) illustrates the angle meter 344. The angle meter 344 has substantially the same configuration and function as the angle meter 324. An overview of the back angle can be known from a display 344a of the angle meter 344.

As illustrated in FIG. 12 (c), the switch portion 347 is disposed on the inner surface 340G (see FIG. 9) of the head left side rail 340. The switch portion 347 has substantially the same configuration and function as the switch portion 327. The switch portion 347 includes switches 347a to 347d. The switches 347a to 347d have substantially the same functions as the switches 327a to 327d. A switch 347n and a USB terminal 347u are disposed on the inner surface 320G. The USB terminal 347u may be omitted.

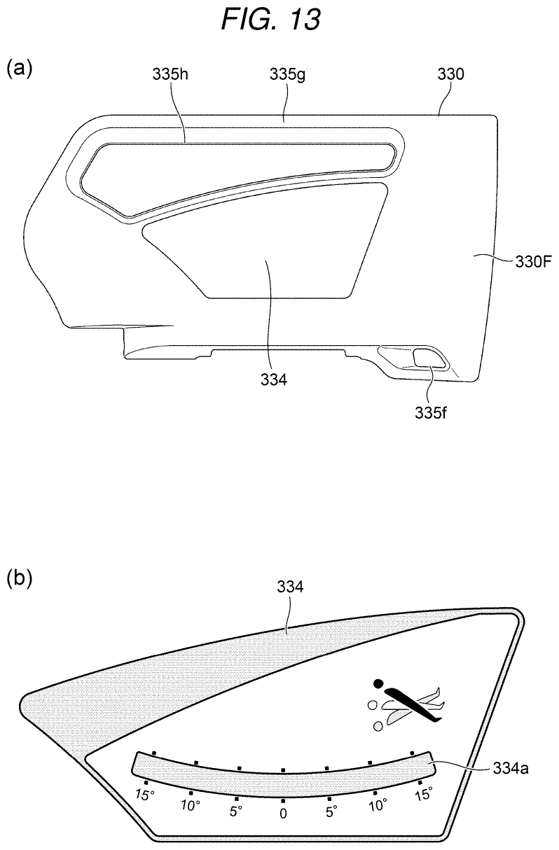

FIG. 13(a) and FIG. 13(b) are schematic diagrams illustrating part of the bed apparatus according to the embodiment.

As illustrated in FIG. 13(a), the foot right side rail 330 is provided with the handrail 335g. The handrail 335g is formed by a through hole 335h. A lower through hole 335f (e.g., a Harn bag hook) is formed in a lower portion of the foot right side rail 330. A Harn bag, for example, can be hung from the lower through hole 335f.

An angle meter 334 is disposed on the outer surface 330F of the foot right side rail 330 (see FIG. 13(b)). The angle meter 334 has substantially the same configuration as the angle meter 324. An overview of an angle can be known from a display 334a of the angle meter 334.

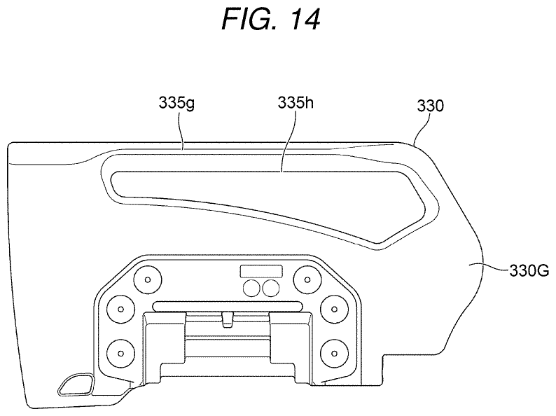

FIG. 14 is a schematic diagram illustrating part of the bed apparatus according to the embodiment.

As illustrated in FIG. 14, the through hole 335h is formed in the foot right side rail 330 so as to acquire the handrail 335g.

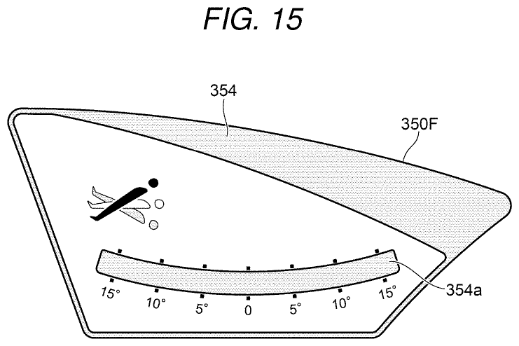

FIG. 15 is a schematic diagram illustrating part of the bed apparatus according to the embodiment.

FIG. 15 illustrates an angle meter 354 disposed on the outer surface 350F of the foot left side rail 350. The angle meter 354 has substantially the same configuration as the angle meter 324. An overview of an angle can be known from a display 354a of the angle meter 354.

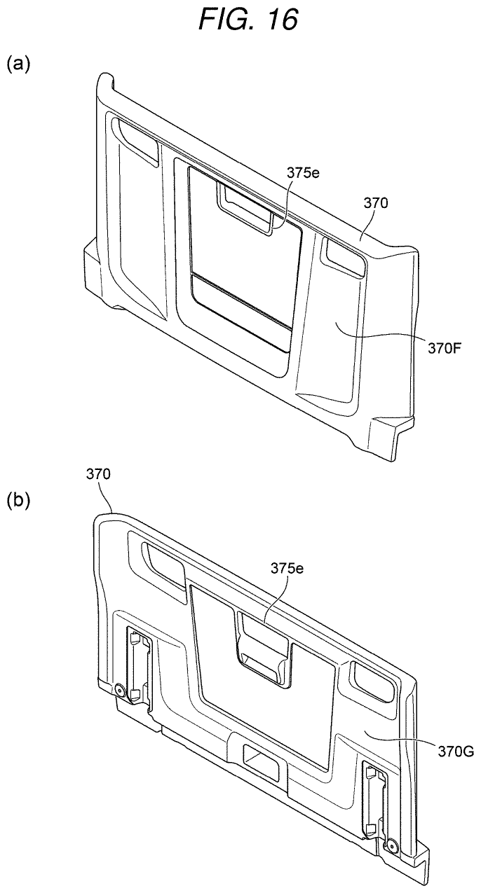

FIG. 16(a) and FIG. 16(b) are schematic diagrams illustrating part of the bed apparatus according to the embodiment.

As illustrated in FIG. 16(a), a through hole 375e (e.g., a hook portion) is formed in the outer surface 370F of the footboard 370. The bed user interface device 380 can be hung from the through hole 375e. As illustrated in FIG. 16(b), the through hole 375e extends through to an inner surface 370G of the footboard 370.

In the bed user interface device 380, various kinds of setting concerning the bed 310B and the weight of the user can be displayed. As a “physical button” in the bed user interface device 380, a “home button” is provided. An example of the bed user interface device 380 will be described later.

FIG. 17 (a) to FIG. 17 (c) are schematic perspective views illustrating operation of the bed apparatus according to the embodiment.

These drawings illustrate states where the mattress 390M is not provided.

As illustrated in FIG. 17 (a), in the bed 310B, the frame 390F is attached to a base frame 390B. The back section 70a (back section), the upper leg section 70b (upper leg section), and the lower leg section 70c (lower leg section), for example, are disposed on the frame 390F. In this example, a seat section 70e is provided. The base frame 390B may be provided with casters 390C.

As illustrated in FIG. 17(a), in the bed apparatus 310, an angle (an inclination) of the frame 390F is variable. The inclination may include lateral inclination in addition to fore-and-aft inclination.

As illustrated in FIG. 17(b), in the bed apparatus 310, an angle of each of the back section 70a, the upper leg section 70b, and the lower leg section 70c is variable. Angles of the head right side rail 320 and the head left side rail 340 change in accordance with a change in angle of the back section 70a. The head right side rail 320 and the head left side rail 340 are follow-up side rails. A state of FIG. 17(b) corresponds to the cardiac position.

In an example of FIG. 17(b), the foot right side rail 330 and the foot left side rail 350 are in “raised states”.

As illustrated in FIG. 17(c), the foot right side rail 330 and the foot left side rail 350 can shift to “lowered states”.

As illustrated in FIG. 17 (b) and FIG. 17(c), the height of the bed 310B is variable. The height corresponds to, for example, a distance between an upper surface of the bed 310B (e.g., upper surfaces of the sections) and the floor surface.

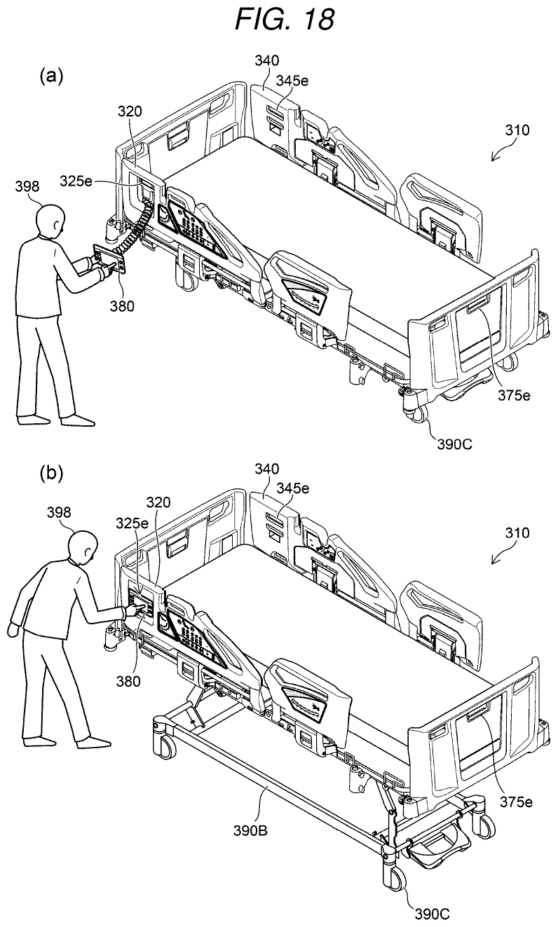

FIG. 18(a) and FIG. 18 (b) are schematic perspective views illustrating use states of the bed apparatus according to the embodiment.

FIG. 18(a) illustrates a low state of the bed 310B. At this time, a care worker or the like 398 (e.g., a care worker or a medical worker) can operate the bed user interface device 380 in a state detached from the hook portion (e.g., the through hole 325e of the head right side rail 320).

FIG. 18 (b) illustrates a high state of the bed 310B. At this time, the care worker or the like 399 can operate the bed user interface device 380 in a state attached to the hook portion. The bed user interface device 380 may be attached to, for example, three hook portions. The three hook portions are the through hole 325e of the head right side rail 320, the through hole 345e of the head left side rail 340, and the through hole 375e of the footboard 370.

Hereinafter, exemplary electrical components in the bed apparatus 310 will be described.

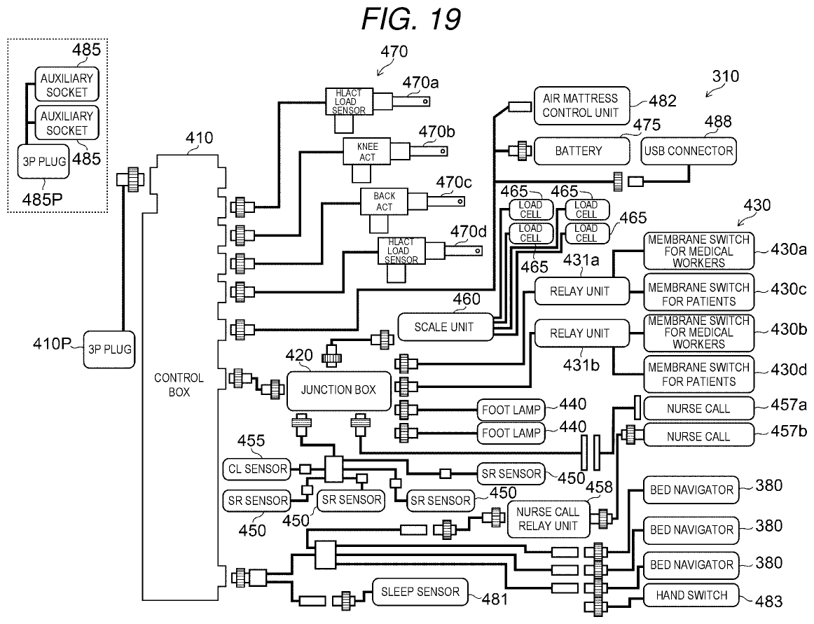

FIG. 19 is a schematic diagram illustrating the bed apparatus according to the embodiment.

As illustrated in FIG. 19, a control box 410 is disposed in the bed apparatus 310. In addition to this, various devices are disposed in the bed apparatus 310. The various devices include, for example, a junction box 420, a membrane switch 430, foot lamps 440, side rail sensors 450 (SR sensors), a caster lock sensor 455 (a CL sensor), a nurse call 457a, a nurse call 457b, a nurse call relay unit 458, a scale unit 460, load cells 465, an actuator 470, and a battery 475. Some of the various devices may be omitted.

The control box 410 is connectable to the various devices. Connection between the control box 410 and the various devices is direct or via the junction box 420. The control box 410 controls bed operation and various functions. The control box 410 serves as a master of serial communication in the bed apparatus 310.

The control box 410 is provided with a plug 410P (e.g., 3-pin plug). Power is supplied from the plug 410P to the control box 410. Power is supplied from the control box 410 to the various devices.

The junction box 420 relays connection between the control box 410 and the rest of the various devices.

The membrane switch 430 includes membrane switches for medical workers 430a and 430b. These membrane switches correspond to the switches 323 and 343. The membrane switch 430 includes membrane switches for patients 430c and 430d. These membrane switches correspond to the switches 327 and 347. The membrane switch for medical workers 430a and the membrane switch for patients 430c are connected to the junction box 420 via a relay unit 431a. The membrane switch for medical workers 430b and the membrane switch for patients 430d are connected to the junction box 420 via a relay unit 431b.

The membrane switches for medical workers 430a and 430b are provided with bed operation buttons (switches). As already described, the bed operation buttons include, for example, a cardiac operation button (such as a conjunctional operation button), an expansion/reverse expansion button, a kind motion operation button (e.g., another conjunctional operation button), a CPR button, a nurse call button, a membrane switch for patients inhibition button, and an all switch inhibition button. With these bed operation buttons, operation described concerning the switch portions 323 and 343 is performed.

The membrane switches for patients 430c and 430d are provided with bed operation buttons. As already described, the bed operation buttons include, for example, aback section operation button, an upper leg section operation button, and a nurse call button. The membrane switches for patients 430c and 430d may include, for example, charging terminals.

In the membrane switches for medical workers 430a and 430b and the membrane switches for patients 430c and 430d, the bed operation buttons are disposed at intermediate positions in the vertical direction. When the bed operation buttons are disposed at lower positions, the bed operation buttons are difficult to operate. When the bed operation buttons are disposed at upper positions, the bed operation buttons may be erroneously operated. Since the bed operation buttons are disposed at intermediate positions in the vertical direction, the bed operation buttons can be easily operated and prevented from being erroneously operated.

Foot lamp buttons correspond to the switches 323q and 343q. When the foot lamp buttons are depressed, the foot lamps 440 are lit. The foot lamps 440 illuminate, for example, end portions of the bed 310B to the floor surface. The foot lamps 440 are provided, for example, one on each of the left and right sides of the bed 310B. The foot lamps 440 are disposed, for example, on left and right end portions of a rear side (a lower side) of the seat section 70e. The foot lamps 440 may be disposed on other portions, such as the back section 70a, the upper leg section 70b, and the lower leg portion 70c (see, for example, FIG. 17 (b)). For example, each time the foot lamp buttons (the switches 323q and 343q) are depressed, the foot lamps 440 are unlit, lit dimly, lit brightly, and unlit in sequence repeatedly. For example, the foot lamps 440 are lit by a medical worker. For example, when the user goes to a toilet highly frequently, a medical worker turns on the foot lamps 440 before the user falls asleep. For example, when bed departure is detected or when a nurse call is dispatched, a medical worker turns on the foot lamps 440.

For example, when the user of the bed 310B leaves the bed for a toilet or some other place at night, the foot lamps 440 are lit. At this time, it is dark in the room in many cases. When the foot lamps 440 are abruptly lit brightly, brightness will be a nuisance to those who are asleep in the vicinity. When the foot lamps 440 are first lit dimly, the nuisance is suppressed.

The side rail sensors 450 detect whether the respective side rails are raised. The four side rail sensors 450 are provided. The four side rail sensors 450 include a head right side rail sensor, a head left side rail sensor, a foot right side rail sensor, and a foot left side rail sensor. A detection result is displayed, for example, in a terminal at a nurses' station. The detection result may be displayed in the bed user interface device 380. Based on the detection result, a warning beep may sound. As the side rail sensors 450, for example, magnetic sensors or atmospheric sensors are employed. As the side rail sensors 450, other sensors may be employed.

The caster lock sensor 455 detects whether the casters 390C are locked. As the caster lock sensor 455, for example, a magnetic sensor is employed. For example, the casters 390C are provided with bars or the like that operate in conjunction with locking and lock release of the casters 390C. States of the bars are detected to detect lock states of the casters 390C. A detection result by the caster lock sensor 455 is displayed, for example, in a terminal at a nurses' station. The detection result may be displayed in the bed user interface device 380. Based on the detection result by the caster lock sensor 455, a warning beep may sound.

The nurse call 457a is connected to the junction box 420. The nurse call 457b is connected to the nurse call relay unit 458. The nurse call relay unit 458 enables conjunctional operation with nurse calls (e.g., the nurse call 457b) disposed in a hospital and a facility. The nurse calls 457a and 457b are nurse calls produced domestically or overseas. For example, the nurse call 457a is a foreign product. For example, the nurse call 457b is a domestic product.

The load cells 465 are disposed at four corners of the bed 310B. The four load cells 465 are used. The load cells 465 and the scale unit 460 can measure the weight of the user.

The actuator 470 includes, for example, an actuator 470a for height change (“HLACT”), an actuator 470b for the upper leg section 70b (“knee ACT”), an actuator 470c for the back section 70a (“back ACT” with the CPR), and an actuator 470d for height change (“HLACT”). The actuators 470a and 470d include load sensors.

In one example, the actuator 470c for the back section 70a includes a mechanical mechanism to manually perform lowering operation (hereinafter referred to as manual CPR mechanism). With the manual CPR mechanism, in an emergency, the back section 70a can be manually lowered. For example, an exclusive-use lever is provided, and this lever is operated to manually lower the back section 70a so that a posture for the CPR can be acquired. For example, manually, a brake plate of the actuator 470c for the back section 70a can be displaced. Thus, a brake of the actuator 470c is released, and the back section 70a is lowered under its own weight.

The actuators 470 become a drive source to adjust movable parts that the bed 310B includes. The actuators 470 move extendable rods to operate the movable parts through a link mechanism, for example. Each of the actuators is provided with a position sensor. The control box 410 reads positional information. The load sensors of the actuators 470 may determine a movement (including bed departure, for example) by the user (e.g., a patient) on the bed 310B.

The battery 475 supplies power at the time of power breakdown or during transfer of the bed 310B, for example. In a situation without power supply, a desired operation can be acquired. A change switch may be provided to charge or not charge the battery 475. Irrespective of a state of the change switch, charge may be possible insofar as power (AC power source) is supplied to the bed 310B.

For example, when the bed apparatus 310 is driven by an AC power source, power is supplied from the control box 410 to the battery 475, an air mattress control unit 482, and a USB charger 488 (see FIG. 19). When no power is supplied from the AC power source, the battery 475 supplies power to the control box 410, the air mattress control unit 482, and the USB charger 488. When no power is supplied from the AC power source, and no power is supplied from the battery 475, the bed 310B is not operated.

As illustrated in FIG. 19, the bed apparatus 310 is provided with a sleep sensor 481, the air mattress control unit 482, and the bed user interface device 380. The bed apparatus 310 may be provided with a hand switch 483.

The sleep sensor 481 measures a sleeping status of the user (e.g., a patient) of the bed 310B. When the bed user interface device 380 is provided, a measurement result of the sleep status and a sleep history may be output to (e.g., displayed in) the bed user interface device 380.

For example, the control box 410 is provided with a connector for the air mattress control unit 482. Conjunctional operation of an air mattress may be performed in accordance with a posture of the bed 310B. The conjunctional operation may differ in accordance with a kind of the air mattress. Setting and changes of operation of the air mattress may be performed by the bed user interface device 380.

The bed apparatus 310 is further provided with auxiliary sockets 485. In this example, the two auxiliary sockets 485 are provided. The auxiliary sockets 485 are plug receptacles. The auxiliary sockets 485 include a plug 485P. The plug 485P is a plug that satisfies medical standards. The plug 485P is a 3-pin plug. The plug 485P is provided as a different component from the plug 410P of the control box 410.

The bed apparatus 310 may include the USB charger 488 (see FIG. 19). The USB charger 488 corresponds to the USB terminal 327u (or 347u). The USB charger 488 feeds power to devices that allow USB charge. The number of ports of the USB charger 488 may be 1. An output rating of the USB charger 488 is DC5V/1A. The port is disposed in the membrane switch for patients 430c of the right side rail.

The bed apparatus 310 may include an error display LED. The error display LED corresponds to the indicators 323t and 343t.

In the bed apparatus 310, bed departure of the user of the bed 310B may be detected. For example, the bed departure is detected by the load cells 465. For example, the bed departure is detected by load sensors incorporated in the actuators. Information concerning the bed departure is transmitted to the nurse call system and output to a terminal at the nurses' station. The information concerning the bed departure may be output to the bed user interface device 380. The output of the information concerning the bed departure may include, for example, visual stimulus such as a lamp or audio stimulus such as a warning beep.

Hereinafter, an example of the bed user interface device 380 will be described.

The bed user interface device 380 is connected to the bed 310B. With the bed user interface device 380, setting concerning the bed 310B can be performed and displayed. Languages of display in the bed user interface device 380 can be changed. For example, display in Japanese, English, Chinese or Portuguese is possible. The bed user interface device 380 is attached to, for example, the left and right side rails or the footboard 370.

The maximum number of the bed user interface devices 380 disposed on the bed apparatus 310 is, for example, 3. In one example, the single bed user interface device 380 or the single hand switch 483 (described later) is connected to the bed 310B. In another example, the single bed user interface device 380 and the single hand switch 483 are connected to the bed 310B. In another example, the two bed user interface devices 380 are connected to the bed 310B. In another example, the two bed user interface devices 380 and the single hand switch 483 are connected to the bed 310B. In another example, the three bed user interface devices 380 are connected to the bed 310B.

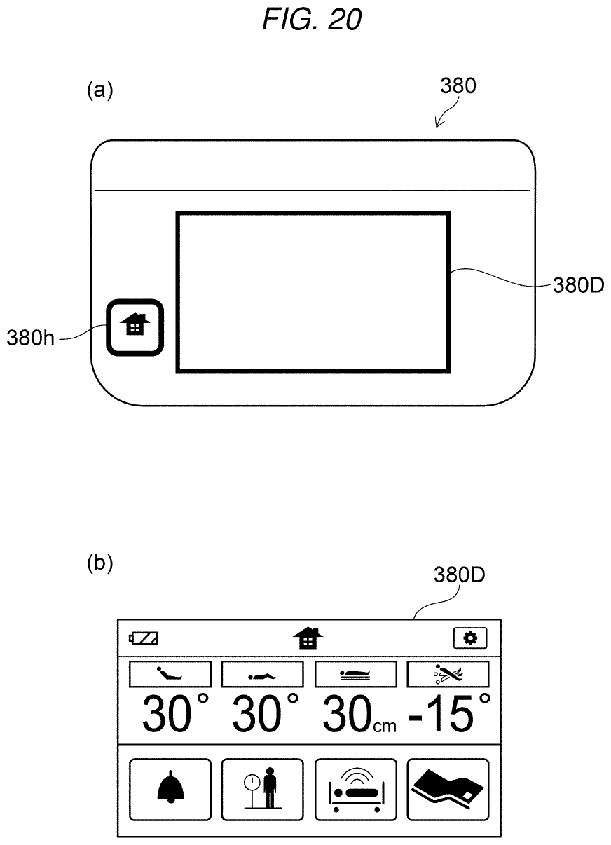

FIG. 20(a) and FIG. 20(b) are schematic diagrams illustrating part of the bed apparatus according to the embodiment.

FIG. 20(a) illustrates the bed user interface device 380 mainly disposed on the head side rail (the head right side rail 320 or the head left side rail 340). The bed user interface device 380 includes a display and input portion 380D. The bed user interface device 380 is provided with a home button 380h.

As illustrated in FIG. 20(b), various kinds of display in the display and input portion 380D are possible. A posture of the bed 310B and the weight of the user can be displayed in the display and input portion 380D. With the display and input portion 380D, setting of the bed departure sensor is possible. Display concerning the sleep sensor 481 can be performed in the display and input portion 380D. With the display and input portion 380D, the air mattress can be operated. Error display can be performed by the display and input portion 380D.

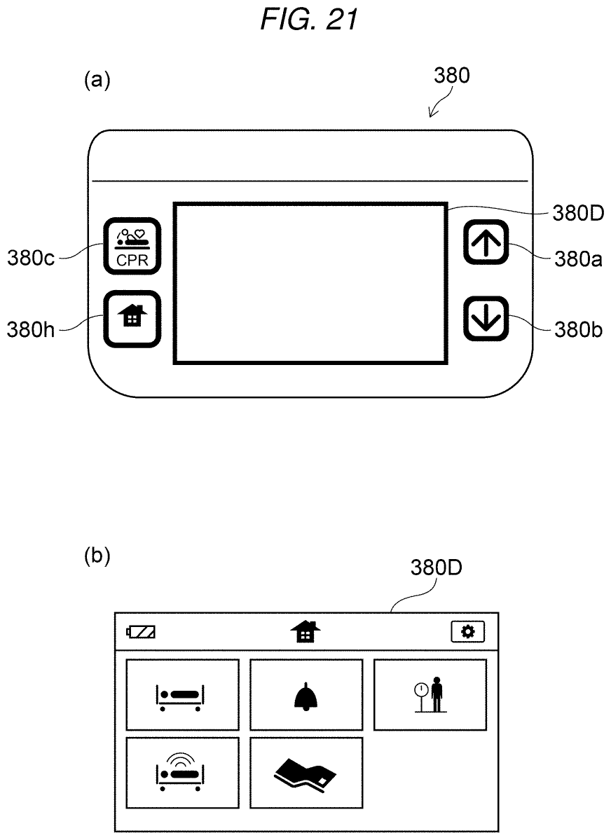

FIG. 21(a) and FIG. 21(b) are schematic diagrams illustrating part of the bed apparatus according to the embodiment.

FIG. 21(a) illustrates the bed user interface device 380 mainly disposed on the footboard 370. The bed user interface device 380 includes the display and input portion 380D. In the bed user interface device 380, a raising button 380a, a lowering button 380b, and a CPR button 380c are provided in addition to the home button 380h. The raising button 380a or the lowering button 380b causes a movable part of the bed 310B to be raised or lowered. The CPR button 380c causes the bed 310B to shift to a posture for the CPR.

As illustrated in FIG. 21(b), various kinds of displays can be performed in the display and input portion 380D. The bed 310B can be operated by the display and input portion 380D. Examples of bed operation include cardiac operation, inclination operation, conjunctional operation (kind operation), back raising and lowering, knee raising and lowering, and height raising and lowering. The weight of the user can be displayed by the display and input portion 380D. Setting of the bed departure sensor can be performed by the display and input portion 380D. Display concerning the sleep sensor 481 can be performed by the display and input portion 380D. The air mattress can be operated by the display and input portion 380D. Error display can be performed by the display and input portion 380D.

Hereinafter, an example of the hand switch 483 will be described.

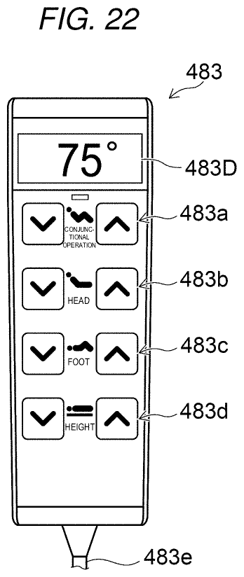

FIG. 22 is a schematic diagram illustrating part of the bed apparatus according to the embodiment.

FIG. 22 illustrates the hand switch 483. The hand switch 483 includes switch pairs 483a to 483d. The switch pair 483a includes switches for raising or lowering concerning “conjunctional” operation. The switch pair 483b includes switches for raising or lowering concerning “back raising” operation. The switch pair 483c includes switches for raising or lowering concerning “foot raising” operation. The switch pair 483d includes switches for raising or lowering concerning “height” change operation.

An angle or a height may be displayed in a display portion 483D of the hand switch 483. The hand switch 483 is connected to the control box 410, for example, via a component such as a cable 483e.

Hereinafter, an example of the auxiliary socket 485 will be described.

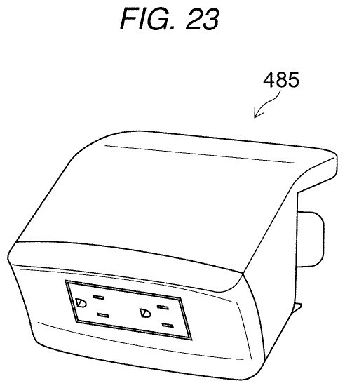

FIG. 23 is a schematic perspective view illustrating part of the bed apparatus according to the embodiment.

FIG. 23 illustrates the auxiliary socket 485 (e.g., plug receptacle device). A plug of an electronic appliance or the like used in the vicinity of the bed 310B can be connected to the auxiliary socket 485. As already described, the plug 485P of the auxiliary socket 485 is provided as a different component from the plug 410P of the control box 410. The auxiliary socket 485 includes two sets of plug receptacles (plug insertion holes). The two sets of plug receptacles are disposed side by side.

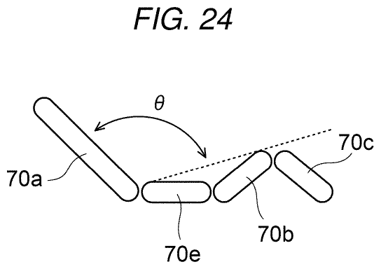

FIG. 24 is a schematic diagram illustrating part of the bed apparatus according to the embodiment.

FIG. 24 illustrates the back section 70a, the upper leg section 70b, the lower leg section 70c, and the seat section 70e. An angle of each of the back section 70a, the upper leg section 70b, and the lower leg section 70c is variable. Angles between the sections are controlled not to be equal to or less than a predetermined value (e.g., 90 degrees). For example, an angle between a line connecting a lower end of the back section 70a and an upper end of the upper leg section 70b (a dashed line in FIG. 24), and the back section 70a is controlled to be equal to or less than the predetermined value (e.g., 90 degrees). For example, control (conjunctional operation) of movements of some of the plurality of sections causes the angles to be equal to or less than the predetermined value.

An operation angle of the back section 70a is, for example, 0 degree to 70 degrees. An operation angle of the upper leg section 70b is, for example, equal to or larger than 0 degree and equal to or less than 25 degrees. An operation range of the “height” is, for example, 43 cm. The bed height may be different in accordance with the bed frame. A range of the bed height is, for example, 30 cm to 73 cm, 32.5 cm to 75.5 cm, or 35 cm to 78 cm.

An operation angle of inclination of the sections is −15 degrees to 15 degrees. For example, to avoid interference in the bed frame, an inclination operation is performed after the height is adjusted to a “minimum bed height+3 cm”.

Operation of the bed 310B to shift to the cardiac position and operation of the bed 310B to make the sections flat are accompanied with the inclination operation. An operation sequence considering slippage of the user is applied to these operations.

In the electric CPR operation, operations are performed in the following sequence. At the time, when simultaneous operations are possible, operations may be performed simultaneously. In the electric CPR operation, the actuator 470c (“back ACT”) for the back section 70a is first operated to a lower limit of its stroke. Within 30 seconds after a button for the electric CPR operation is depressed, the actuator 470c reaches the lower limit. Thereafter, inclination operation is performed to make an inclination angle 0 degree. Thereafter, height adjustment is performed to make the height the minimum bed height. The minimum bed height is, for example, a “temporary pause height”. Thereafter, the upper leg section 70b is operated to be at 0 degree.

In the bed apparatus 310, operation of the bed user interface device 380 can cause button operations relating to various kinds of operation to be in an “operation inhibited” state.

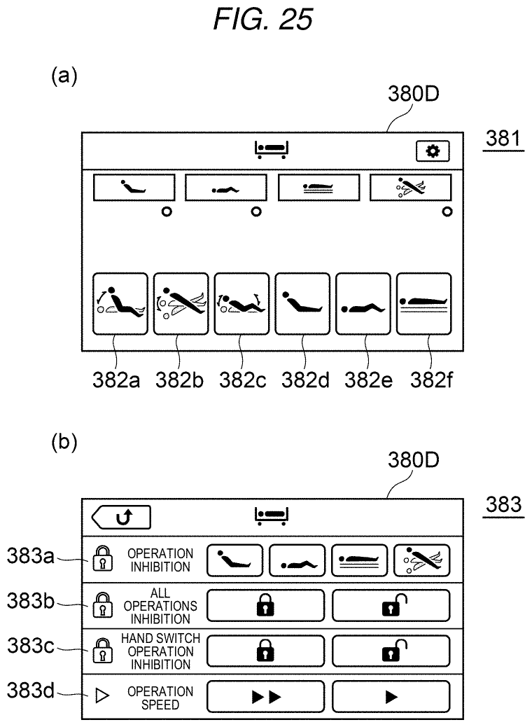

FIG. 25(a) and FIG. 25(b) are schematic diagrams illustrating part of the bed apparatus according to the embodiment.

These drawings illustrate the display and input portion 380D of the bed user interface device 380. FIG. 25(a) illustrates a case where the display and input portion 380D is a bed user interface display screen 381. FIG. 25(b) illustrates a case where the display and input portion 380D is an actuator individual operation inhibition display screen 383 (a bed setting display screen).

As illustrated in FIG. 25(a), for example, buttons 382a to 382f are provided. These buttons are, for example, a user interface input region in a touch input device. These buttons are touched to perform bed operation. Cardiac operation is performed by the button 382a. Inclination operation is performed by the button 382b. Conjunctional operation (“kind operation”) is performed by the button 382c. Back operation (back angle change) is performed by the button 382d. Knee operation (knee angle change) is performed by the button 382e. Height operation (height change) is performed by the button 382f. For example, in the case of raising the back of the bed 310B, the button 382d is depressed, and thereafter, the raising button 380a so as to move the bed 310B. The bed 310B is moved while the raising button 380a is being depressed. For example, in the case of lowering the height of the bed, the button 382f is depressed, and thereafter, the lowering button 380b so that the height of the bed 310B is lowered while the lowering button 380b is being depressed.

As illustrated in FIG. 25 (b), the display and input portion 380D can shift to the actuator individual operation inhibition display screen 383 (the bed setting display screen). When a display and input region 383a is operated, each of the back operation, the knee operation, the height operation, and the inclination operation can be individually inhibited. When a display and input region 383b is operated, all the operations can be inhibited, or operation inhibition can be released. When a display and input region 383c is operated, operation of the hand switch 483 can be inhibited or operation inhibition can be released.

For example, when the back operation is inhibited, the cardiac operation, the conjunctional operation, and the back operation are inhibited. For example, when the knee operation is inhibited, the cardiac operation, the conjunctional operation, and the knee operation are inhibited. For example, when the height operation is inhibited, the cardiac operation, the inclination operation, and the height operation are inhibited. For example, when the inclination operation is inhibited, the cardiac operation and the inclination operation are inhibited.

In one example, “all operations inhibition” is possible. In another example, “operation inhibition” of the actuators 470 (e.g., 470a to 470d (see FIG. 19)) may be selectively (individually) performed. The “operation inhibition” of the actuators 470 is released by the bed user interface device 380.

When the bed user interface device 380 is detached from the bed 310B or when a cable to connect the bed user interface device 380 breaks, for example, the selective “operation inhibition” of the actuators 470 cannot be released. In this case, the “operation inhibition” of the actuators 470 can be released by releasing the “all operations inhibition”.

The operation inhibition of the actuators 470, the operation inhibition of the hand switch 483 or the “all operations inhibition” is independently controlled. For example, in the case of individual operation inhibition of the actuators 470, the “all operations inhibition” is performed. Thereafter, even when the “all operations inhibition” is released, the individual operation inhibition of the actuators 470 remains.

When an inhibited button is depressed, a buzzer sounds, and an inhibition LED (e.g., indicators 323t and 343t) of the membrane switch flashes on and off. When no buzzer sounds, it is not clear whether the button is inhibited or broken. When a buzzer sounds, it can be made clear that the button is inhibited.

When an inhibited button in the hand switch 483 is depressed, the hand switch 483 rumbles. An inhibited button in the membrane switch is depressed, the junction box 420, for example, rumbles.

Operation of the membrane switch for medical workers or the bed user interface device 380 can make the membrane switch for patients and the hand switch 483 shift to an operation inhibited state. The operation inhibition can be released insofar as the membrane switch for medical workers or the bed user interface device 380 is connected.

Operation of the membrane switch for medical workers or the bed user interface device 380 makes it possible to inhibit all operations (“all operations inhibition”). This operation inhibition can be released insofar as one of the membrane switch for medical workers and a bed navigator is connected.

For example, when the bed user interface device 380 is not connected to the bed 310B or has partial malfunction (establishes no communication), the “all operations inhibition” is released. In this case, for example, the operation can be performed by the hand switch 483. When the user is hindered from operation, the hand switch 483 may be detached. When an inhibited button is depressed, a buzzer sounds, and the inhibition LED of the membrane switch flashes on and off.

When the “all operations inhibition” button is depressed in an operation inhibited state of the hand switch 483, the “all operations inhibition” is established. Thereafter, when the “all operations inhibition” is depressed, the operation inhibition of the hand switch 483 and the all operations inhibition are released. During the “all operations inhibition”, the CPR operation is performed. In any of the “operation inhibition”, the “hand switch operation inhibition”, and the “all operations inhibition”, the CPR operation is performed.

For example, in a “present setting”, in the case where the hand switch 483 is not set in operation inhibition, and the “all operations inhibition” is not set, and when the operation inhibition button in the hand switch 483 is depressed, the hand switch 483 shifts to operation inhibition, and “all the operations” are not inhibited (a released state).

For example, in a “present setting”, in the case where the hand switch 483 is not in operation inhibition, and the “all operations inhibition” is not set, and when the “all operations inhibition” button is depressed, the hand switch 483 and all the operations are inhibited.

For example, in a “present setting”, in the case where the hand switch 483 is in operation inhibition, and the “all operations inhibition” is not set, and when the “operation inhibition” button in the hand switch 483 is depressed, the hand switch 483 is not in operation inhibition (a released state), and “all the operations” are not in operation inhibition (a released state).

For example, in a “present setting”, in the case where the hand switch 483 is in operation inhibition, and the “all operations inhibition” is not set, and when the “all operations inhibition” button is depressed, the hand switch 483 and all the operations are inhibited.

For example, in a “present setting”, in the case where the hand switch 483 is in operation inhibition, and the “all operations inhibition” is set, and when the operation inhibition button in the hand switch 483 is depressed, the hand switch 483 and all the operations are inhibited.

For example, in a “present setting”, in the case where the hand switch 483 is in operation inhibition, and the “all operations inhibition” is set, and when the “all operations inhibition” button is depressed, the hand switch 483 is not in operation inhibition (a released state), and all the operations are not inhibited (a released state).

Operation of the display and input region 383d illustrated in FIG. 25(b) makes it possible to change an operation speed. For example, speeds of various kinds of operation can be changed into a plurality of stages (e.g., 2 stages).

A history relating to various kinds of operation of the bed apparatus 310 may be preserved. The history is preserved in a memory in the control box 410, for example. The memory where the history is preserved may be provided in the junction box 420 or the hand switch 483, for example. The memory where the history is preserved may be provided in the bed user interface device 380. Information concerning the history is not reset by turning on or off the power source. The information concerning the history includes, for example, an operation history of the control box 410, an operation history of the actuators 470, an operation history of the hand switch 483, a history of operation details, a malfunction history, and a bed departure and presence history.

According to the embodiments, it is possible to provide the bed apparatus that can prevent erroneous release of the lock.

Heretofore, with reference to specific examples, the embodiments of the invention have been described. However, the invention is not to be limited to these specific examples. For example, specific configurations of the elements, such as the frame, the side rail, the holder, the lock portion, the cover portion, the lever portion, and the stopper portion of the bed apparatus, fall within the scope of the invention insofar as the specific configurations are appropriately selected from a known scope by those skilled in the art so that the invention can be likewise implemented, and that substantially the same effects can be acquired.

Moreover, when any two or more elements in each of the specific examples are combined in a technically possible range, the combination also falls within the scope of the invention insofar as the combination encompasses the subject matter of the invention.

In addition, all bed apparatuses that those skilled in the art can implement by appropriate design change based on the bed apparatuses described above as the embodiment of the invention belong to the scope of the invention insofar as the bed apparatuses encompass the subject matter of the invention.

Furthermore, within the soul and spirit of the invention, those skilled in the art can easily conceive various modifications and alterations, and it is to be understood that these modifications and alterations also belong to the scope of the invention.

### DESCRIPTION OF REFERENCE NUMERALS AND SIGNS

50, 50A . . . holder, 51 . . . lock portion, 51A . . . hook, 51P . . . pin, 52 . . . cover portion, 52a . . . recess, 52h . . . hole, 53 . . . lever portion, 54 . . . stopper portion, 54H . . . hole, 54Ha . . . first portion, 54Hb . . . second portion, 54P . . . pin, 54p . . . bar-shaped member, 54q . . . first member, 54r . . . second member, 54x . . . shaft, 58 . . . lock component, 70a . . . back section, 70b . . . upper leg section, 70c . . . lower leg section, 70e . . . seat section, 72, 72A . . . side rail, 75 . . . frame, 75B . . . base frame, 78A . . . headboard, 78B . . . footboard, 78M . . . mattress, 110 to 114 . . . bed apparatus, SLK . . . locked state, SP1, SP2, first position state, second position state, SRS . . . released state, ST1, ST2 . . . first state, second state, SUL . . . unlocked state, SUR . . . non-released state, 310 . . . bed apparatus, 310B . . . bed, 320 . . . head right side rail, 320F . . . outer surface, 320G . . . inner surface, 323 . . . switch portion, 323a to 323q . . . switch, 323r to 323t . . . indicator, 324 . . . angle meter, 324a . . . display, 325a . . . protrusion, 325b . . . recess, 325c . . . head-side protrusion, 325d . . . head-side recess, 325e . . . through hole, 325f . . . lower through hole, 325g . . . handrail, 325h . . . through hole, 327 . . . switch portion, 327a to 327d . . . switch, 327n . . . switch, 327u . . . USB terminal, 328 . . . recess, 328h . . . hole, 330 . . . foot right side rail, 330F . . . outer surface, 330G . . . inner surface, 334 . . . angle meter, 334a . . . display, 335f . . . lower through hole, 335g . . . handrail, 335h . . . through hole, 340 . . . head left side rail, 340F . . . outer surface, 340G . . . inner surface, 343 . . . switch portion, 343a to 343q . . . switch, 343r to 343t . . . indicator, 344 . . . angle meter, 344a . . . display, 345e . . . through hole, 347 . . . switch portion, 347a to 347d . . . switch, 347n . . . switch, 347u . . . terminal, 350 . . . foot left side rail, 350F . . . outer surface, 350G . . . inner surface, 354 . . . angle meter, 354a . . . display, 355g . . . handrail, 360 . . . headboard, 370 . . . footboard, 370F . . . outer surface, 370G . . . inner surface, 375e . . . through hole, 380 . . . bed user interface device, 380D . . . display and input portion, 380a . . . raising button, 380b . . . lowering button, 380c . . . CPR button, 380h . . . home button, 381 . . . bed user interface display screen, 382a to 382f . . . button, 383 . . . actuator individual operation inhibition display screen, 383a to 383d . . . display and input region, 390B . . . base frame, 390C . . . caster, 390F . . . frame, 390M . . . mattress, 398 . . . care worker or the like, 410 . . . control box, 410P . . . plug, 420 . . . junction box, 430 . . . membrane switch, 430a, 430b . . . membrane switch for medical workers, 430c, 430d . . . membrane switch for patients, 431a, 431b . . . relay unit, 440 . . . foot lamp, 450 . . . side rail sensor, 455 . . . caster lock sensor, 457a, 457b . . . nurse call link, 458 . . . nurse call relay unit, 460 . . . scale unit, 465 . . . load cell, 470 . . . actuator, 470a to 470d . . . actuator, 475 . . . battery, 481 . . . sleep sensor, 482 . . . air mattress control unit, 483 . . . hand switch, 483D . . . display, 483a to 483d . . . switch pair, 483e . . . cable, 485 . . . auxiliary socket, 485P . . . plug, 488 . . . charger

1. A bed apparatus comprising:
a frame;
a side rail; and
a holder secured to the frame and configured to hold the side rail to make the side rail switchable between a first position state where the side rail is in a first position and a second position state where the side rail is in a second position lower than the first position,
the holder comprising:
a lock portion configured to lock the side rail in the first position state;
a cover portion configured to cover at least part of the lock portion;
a lever portion configured to be operated to release lock of the lock portion; and
a stopper portion configured to make it difficult for the lever portion to operate to release the lock.

2. The bed apparatus according to claim 1, wherein
the cover portion includes a recess, and
at least part of the stopper portion is in the recess.

3. The bed apparatus according to claim 1, wherein
the cover portion includes a recess, and
at least part of the stopper portion is in the recess and below the lever portion.
